(19) United States
(12) Patent Application Publication
(10) Pub. No.: US 2006/0079491 A1
(43) Pub. Date: Apr. 13, 2006

(54) Method of female hormonal contraception using a fixed extended cycle hormonal preparation containing dienogest and ethinyl estradiol

(76) Inventor: Andreas Sachse, Berlin (DE)

Correspondence Address: STRIKER, STRIKER & STENBY, 103 EAST NECK ROAD, HUNTINGTON, NY, 11743, US

(21) Appl. No.: 11/247,868

(22) Filed: Oct. 11, 2005

Related U.S. Application Data
(60) Provisional application No. 60/617,293, filed on Oct. 8, 2004.

(51) Int. Cl. A61K 31/56

(52) U.S. Cl. 514/170

(57) ABSTRACT

The fixed extended cycle method of female hormonal contraception includes continuous daily administration of daily dosage units of a monophasic preparation to a woman over a time period of n×21 days, wherein n=2, 3, 4 or 5, immediately followed by a seven-day pill-break in which the monophasic preparation is not administered. Each of the daily dosage units contains from 0.5 to 3.0 mg of dienogest and from 10 to 30 μg of ethinyl estradiol. In addition to effective contraceptive action the method significantly reduces the bleeding rate, is simple and is effective in treating acne, dysmenorrhea and/or endometriosis.

### CROSS-REFERENCE

U.S. Provisional Patent Application, Ser. No. 60/617,293, filed Oct. 8, 2004, entitled “Use of Dienogest in Fixed Extended Cycle Hormonal Contraceptives”, describes the invention, which is described in the specification hereinbelow and claimed in the claims appended hereinbelow, and provides the basis for a claim of priority for the instant invention under 35 U.S.C. 119 (e).

### BACKGROUND OF THE INVENTION

1. The Field of the Invention

The present invention relates to a method for female hormonal contraception, which involves use of dienogest in fixed extended cycle hormonal contraceptives to avoid the bleeding problems associated with continuous use of hormonal contraceptives with other progestins and schedules.

2. Related Art

The first large study based on a fixed extended oral contraceptive regimen (i.e. 90 days cycle=84 days of active pills followed by 6 hormone-free (placebo pills)) was published by Loudon and his colleagues in 1977 (British Medical Journal, 1977; 2: 487-490). In this study a monophasic oral contraceptive [50 μg Ethinyl estradiol (EE)/2.5 mg lynestrenol] was used. Despite the high EE dosage intermenstrual bleeding was observed during the extended cycles in a notable number of women, which decreased with each 90-day cycle. Twenty seven percent of the patients experienced intermenstrual spotting and breakthrough bleeding during the first three-month cycle, which decreased to 4% (spotting only) in the fourth extended cycle. Intermenstrual bleeding accounted for 11% of the dropouts from the study.

Seventeen years later a prospective study of continuous use of 30 μg EE+150 μg LNG (NORDETTE®) over 84 days followed by one week of placebo was published by Kovacs, et al (The British Journal of Family Planning, 1994; 19: 274-275). Of the 203 women who entered the study only 59 (29.1%) completed 12 months of treatment (4×84+7 days). The most frequent reasons for discontinuation were breakthrough bleeding in 73 patients (50.7%) and breast tenderness and headaches in 31 patients (21.5%) each. The dropout rate was highest during the first extended cycle (13 weeks) in which 34.5% (n=70) of the enrolled women were lost while the dropout rate in the second to fourth extended cycle (26, 39+52 weeks) amounted to 21.8% (43), 12.3% (25) and 3.0% (6) respectively. The authors stated that even though the ability to decrease the incidence of menstruation was appreciated by many women this was negated to some degree by the high incidence of breakthrough bleeding.

Hodgen disclosed a corresponding fixed regimen for oral contraceptive use, which should maintain efficacy while providing enhanced control of endometrial bleeding (U.S. Pat. No. 5,898,032). In addition to less menstrual bleeding and patient anemia, higher compliance rates and more lifestyle convenience for patients are listed as advantages of this method. According to the claims, a monophasic combination of an estrogen and progestin is continuously administered for 60-110 consecutive days followed by 3-10 days of no administration. The claimed daily amounts of estrogen and progestin are equivalent to 5-35 μg EE and 0.025-10 mg of norethindrone acetate (NETA).

Other progestins like levonorgestrel (LNG), desogestrel (DSG) or 3-keto-desogestrel are also described (not including dienogest). The weight ratio of the two active ingredients (estrogen: progestin) is disclosed to be at least 1:45 and preferably at least 1:50.

A respective fixed extended 91-day OC (oral contraceptive) regimen (84 days active+7 days placebo) was tested for the first time in a phase III multi-center 1-year trial versus a normal 28-day cycle (21 days active+7 placebo pills) by Anderson, et al, (Contraception, 2003; 68: 89-96). The extended cycle regimen (30 μg EE/150 μg LNG each, SEASONALE®, Barr Laboratories) was found to result in a greater number of unscheduled (breakthrough) intermenstrual bleeding amounting to 37.6 days compared to 18.3 days for the 28-day regimen. Most notably the total days of bleeding unscheduled+scheduled (=menstrual bleeding) during the study year (364 days) were 48.2 days for the extended cycle compared to 50.8 days for the standard cycle. It is reported that for the extended cycle breakthrough bleeding (BTB) decreased with each successive cycle (84+7 each) from a median of 12 days during cycle 1 to a median of four days during cycle 4.

The most common reasons given for study discontinuation were bleeding, increased weight, mood swings and acne (=adverse events). Discontinuation due to unacceptable bleeding accounted for 7.7% of the 91-day regimen patients compared to 1.8% in the 28-day regimen group. The total dropout rates amounted to 40.6% and 28.8% respectively. In the Clinical Review of NDA 21-544 (Sep. 4, 2003) for SEASONALE® besides the results for SEASONALE® also results for SEASONALE® Ultra-Lo (20 μg EE/100 μg LNG daily) are reported. It was observed that the fixed (84+7 days) extended regimen with a lower EE dose results in an even worse bleeding control.

In a 12-months randomized multi-center study the acceptance of extended use (63+7 (pill-free) days) of a monophasic oral contraceptive with 30 μg EE and 150 μg desogestrel was studied by Cachrimanidou in comparison to the corresponding standard cycle product (21+7) (Contraception, 1993, 48: 205-216). Breakthrough bleeding was found to be significantly more frequent in the extended cycle (n=198) group compared to the standard cycle group (n=96). Correspondingly more women on the extended cycle product discontinued the study for bleeding problems (13% versus 2%, P<0.01). In both groups intermenstrual bleeding (breakthrough and spotting) decreased over time. Women who were already using oral contraceptives when they started the study had significantly fewer episodes of intermenstrual bleeding compared to starters.

According to the authors a relatively large number of women withdrew from the trial without medical reasons (17.2% extended, 15.6% standard cycle).

Hesch (U.S. Pat. No. 6,500,814) discloses a low dose extended cycle product/regimen, which according to the inventor surprisingly ensures high contraceptive reliability and prevents intermenstrual bleeding. Additionally a reduction in oral contraceptive related side effects (e.g. thrombosis) and a favorable effect on the pre-menstrual syndrome (PMS) are described. Furthermore prophylaxis and treatment of breast cancer are possible with the product according to the invention. Hesch claims continuous and uninterrupted administration of a combined hormonal contraceptive for a period of greater than 110 days. Various natural or synthetic estrogens and progestins (not including dienogest) are described. When EE is used its dosage is claimed to be between 1-20 μg/day.

Kulmann (WO 02/22110) discloses another process for hormonal contraception, which reduces the number of withdrawal bleedings while ensuring reliable contraception. The process is characterized by a sequence of successive extended cycles (=“taking periods”) with increasing duration. Thus for example the patient may start with one taking period of 21 active tablets followed by 7 placebos (21/7) which is followed by a taking period of n×42/7. With the exception of the final taking period the duration of all prior periods is predetermined (fixed). According to the invention it is also possible to successively reduce the hormone dosage (various disclosed progestins including dienogest and/or estrogens) between taking periods.

Sulak, et al. (Am J. Obstet. Gynecol, 2002; 186:1142-1149) retrospectively studied the acceptance of extended cycle use in a larger number of patients with hormone withdrawal symptoms. Patients were allowed to alter their standard 21+7 regimen by extending a specific number of weeks such as 6, 9 or 12 or extending until breakthrough bleeding or spotting developed, stopping for 3-7 days and resuming. If they completed 12 weeks of active pills and wished to continue without a hormone-free break they were allowed to do so. All patients were prior pill users taking monophasic pills with 30-35 μg ethinyl estradiol and one of the following progestins: norethindrone, levonorgestrel, norgestimate or desogestrel. Of the 267 patients who initiated the extended cycle regimen 57 (21%) chose to stop using oral contraceptives for various reasons like worsening of side effects including nausea, headache, acne, leg cramps, high blood pressure, yeast infections, breakthrough bleeding and PMS (24 patients) and a desire for pregnancy (13 patients). Of the 210 patients who continued to use OCs, 38 (18%) chose to return to the standard 21/7 regimen most commonly due to breakthrough bleeding (11 patients), breakthrough spotting (9) and heavy withdrawal bleeding (2 patients).

Zahradnik and Moore (in: Elstein, M., “Extragenital Effects of Oral Contraceptives”, Carnforth: Parthenon Publishers, 1997; pp 53-60) studied the contraceptive efficacy, cycle stability, tolerability and compliance of a new standard cycle (21+7 (tablet-free interval)) contraceptive containing 30 μg EE+2 mg dienogest (VALETTE®). It was found that the severity and quantity of intermenstrual bleeding, spotting and breakthrough bleeding decreased during the course of the one year phase III study (12 cycles). Additionally a pronounced effect on the severity of dysmenorrhea was found which disappeared nearly completely during the study. These effects on bleeding were regarded as highly relevant for the good acceptance of this low-dose oral contraceptive.

Continuous use of VALETTE® (30 μg EE+2 mg dienogest) over 189 days followed by a hormone-free interval of seven days has been studied in 30 women in a private gynecological practice (Wiegratz, et al., Contraception, 2004; 69: 37-42)). Subsequently the women were treated for three standard cycles (21+7 days). Irregular bleeding (BTB+spotting) as well as hormone-related symptoms were recorded in daily diaries.

Irregular bleeding occurred in 40% of the treated women just once while 17% recorded more than two episodes. During the second cycle of treatment (day 21-42) 53% of the women recorded irregular bleeding (mostly spotting) while during the fourth cycle (day 63-84) only 10% still recorded bleeding. Most of the women who had more than two bleeding episodes were first-time users (starters). The lowest bleeding rate was observed during the fifth cycle (3%), which increased to 17% during days 147-168 (cycle 8). Only 50% of the treated women experienced withdrawal bleeding (menstruation) during the 7 day hormone-free interval following the 189 day extended cycle.

All women completed the 189-day extended OC treatment. However, only 18 women (60%) chose to continue the extended regimen while 4 (13%) women each either returned to the normal cycle use or switched to another OC. In the same publication Wiegratz, et al., reported the findings of a survey among German women and gynecologists regarding their attitude towards long-cycle treatment with oral contraceptives. It was found that only 26-35% of the surveyed women between the age of 15 and 49 years wished to have regular menstrual cycles (once a month) while 5-15% preferred 4 menstruations per year. Only 11-14% of women are willing to suppress menstruation continuously or for a prolonged period of time while 32-54% would suppress menstrual bleeding only sporadically. Reasons for not using extended cycle OCs included fear of pregnancy (43-72%) and fear of adverse effects (50-62%).

According to the prior art in the field of extended cycle regimens (see above) it was assumed that stable fixed extended cycles (i.e. acceptable bleeding control) could be obtained and maintained even during the first year of continuous oral contraceptive administration irrespective of the employed progestin. However, in contrast to the claims of Hodgen (U.S. Pat. No. 5,898,032) oral contraceptives with standard progestins do not provide enhanced control of endometrial bleeding compared to the respective standard cycle products during extended cycle use. Thus various independent clinical studies with fixed extended cycle oral contraceptives demonstrated unacceptable bleeding control for standard progestins, for example levonorgestrel (Anderson et al., Kovacs et al.) and desogestrel (Cachrimanidou et al.) disclosed by Hodgen. Most notably very high dropout rates were observed in these studies, mainly due to the bleeding problems (i.e. high rate of intermenstrual bleeding).

Various researchers addressed the problem of intermenstrual bleeding during fixed extended cycle oral contraceptive use. Thus Hesch (U.S. Pat. No. 6,500,814) discloses continuous use of low dose OCs (1-20 μ EE/day) for a period of greater than 110 days, which prevents intermenstrual bleeding. Various progestins (not including dienogest) are claimed.

Kulmann (WO 02/22110), on the other hand, discloses an extended regimen, which is characterized by a sequence of successive fixed extended cycles with increasing duration. This regimen is supposed to circumvent the problems of the fixed extended cycle regimens irrespective of the employed progestin (incl. dienogest).

Finally Sulak, et al., (see above) offered women the option to either extend oral contraceptive use for a specific number of weeks (e.g. 6, 9 or 12) or until intermenstrual bleeding was observed, stopping for 3-7 days and resuming. Monophasic pills with various progestins (norethindrone, levonorgestrel, norgestimate or desogestrel) were employed. Bleeding problems were still found to be the main reason for discontinuation of extended cycle use. The type of oral contraceptive employed was not found to have a significant effect on the continuation of extended OC use.

To date, none of the available extended cycle oral contraceptives (compositions and methods) have demonstrated their suitability to eliminate the bothersome intermenstrual bleeding (breakthrough bleeding and/or spotting) observed during continuous OC use. Thus a significant reduction in the total number of bleeding days compared to the corresponding standard cycle oral contraceptives has not been shown for these products.

### SUMMARY OF THE INVENTION

The object of the present invention is the provision of a method and product for extended oral contraceptive use, which avoids the deficiencies of the available methods and compositions, as reviewed by Henzl and Polan (Journal of Reproductive Medicine 2004; 49:162-174).

First this new method according to the invention should result in a significant reduction of the total number of bleeding days (per year) compared to the corresponding standard cycle oral contraceptive use (superiority regarding bleeding). In some cases the relative number of intermenstrual bleeding days will also significantly be reduced. The improved bleeding pattern should result in a notable decrease in discontinuation (drop-out) rate.

Furthermore the method according to the invention should offer a certain degree of flexibility regarding its duration to allow women to adapt the regimen to their specific biological/medical and individual needs. Such regimen would comply with the desire of the majority of women to reduce the frequency of menstrual bleeding to less than once a month.

Finally this method should offer additional benefits not directly related to bleeding frequency to the patients, e.g. treatment of acne. Surprisingly the problem of intermenstrual bleeding during fixed use of extended hormonal contraceptives in a female can be avoided by a new contraceptive method according to the invention, which comprises continuous daily administration of daily dosage units of a monophasic contraceptive containing dienogest (DNG) and ethinyl estradiol (EE) for contraception over a time period of n×21 days where n=2 to 5 (i.e. 42 to 105 days) followed immediately by a seven-day pill-break in which the monophasic preparation is not administered for seven consecutive days immediately following the time period. The term “immediately followed or following” means that the first day of the seven-day pill-break is the day after the last day of the time period.

### DETAILED DESCRIPTION OF THE INVENTION

More particularly, the method for female hormonal contraception according to the invention comprises continuous administration of daily dosage units of a monophasic preparation of dienogest in a daily amount of 0.5-3.0 mg and ethinyl estradiol in a daily amount of 10 to 30 μ or a synthetic or natural estrogen at a daily dosage equivalent in estrogenic activity to 10 to 30 μ ethinyl estradiol to a woman for a first minimum period of 42 days. After this first minimum period the woman may choose to take a seven-day pill-break (hormone-free period, i.e. no intake of hormones or an intake of placebos), especially if unacceptable bleeding was observed during the previous 21 days, more preferably during the last 7 days. The seven-day pill-break according to the method of the invention will allow the woman to eventually stabilize on the extended cycle schedule so that the maximum extended cycle duration can be achieved without unacceptable intermenstrual bleeding. Alternatively the woman may continue with the extended regimen for multiple periods of 21 days until the maximum cycle duration of 105 days (5×21 days) is reached. At this time the seven-day pill break is mandatory. Between days 42 and 105 the woman on the extended cycle regimen of the present invention may take a voluntary seven-day pill-break after days 63 (3×21) and 84 (4×21) depending on her specific medical/biological situation (e.g. bleeding problems, exclusion of pregnancy)/personal interests/needs (e.g. holidays, sport events, examinations). In preferred cases the maximum extended cycle duration is limited to 84 days (4×21) followed by a seven-day pill-break.

After a pill-break (hormone-free phase) the patient must start over again using the active pills for at least the minimum intake period (i.e. 42 days) before taking the next pill-break. The method according to the invention will result in notably lower dropout rates in clinical settings due to bleeding problems compared to, e. g. SEASONALE®. Consequently, this method will also lead to high compliance and a high acceptance by patients using this method of contraception. The method of the invention will especially allow oral contraceptive starters or switchers to stabilize on the extended cycle OC so that they can eventually extend for the maximum time period (duration) if desired (e.g. 105 days (5×21)).

In the method of the invention the progestin, dienogest (DNG), which exerts an excellent cycle control and also has a pronounced treatment effect on dysmenorrhea, acne and endometriosis, is used. The preferred daily dose is 0.5 to 3.0 mg, more preferably 1.0 to 2.5 mg and most preferably 2.0 mg.

The preferred estrogen is ethinyl estradiol (EE) in a daily dose of 10 to 30 μg, more preferably 20 to 30 μ and most preferably 30 μg. Estradiol and its esters can also be used as the estrogen. The daily amount of estradiol to be administered is 0.5 to 3 μg, preferably 1 to 2 μg. Additionally synthetic estrogens can be used at dosage equivalent to 10 to 30 μg ethinyl estradiol (i.e. with regard to inhibition of gonadotropins and ovulation as well as proliferative effects on the endometrium and vaginal epithelium).

In the method of the invention the employed product will be formulated and administered conventionally, i.e. all standard routes of application, including the various known types of drug preparations (delivery systems), e. g. transdermal patches, IUSs and vaginal rings, and other formulations that are useful for hormonal contraception can be used to perform the invention.

The oral administration route is preferred.

Advantages of the contraceptive method according to the invention include:

achievement of significantly improved bleeding rate (=reduction of total number of bleeding days) compared to a standard 28-day cycle (e.g. 21+7) product, even during the first year of administration, and

simple regimen with weekly schedule (always same starting day) and/or

improved compliance/reduced rate of discontinuation, and/or

reduction in side effects (e.g. nausea, headache bleeding), and/or treatment of acne, dysmenorrhea and/or endometriosis

improvement of skin disorders, and/or

improved quality of life.

Compared to other extended cycle oral contraceptives the product according to the method of the invention is especially useful for the treatment of acne, dysmenorrhea and endometriosis. The flexible schedule of use (2-5×21 days) allows the women to adapt the regimen (i.e. maximum duration) to their specific medical/biological situation (e.g. bleeding problems, exclusion of pregnancy)/personal interests/needs (e.g. holidays, sport events, examinations). This flexibility is especially advantageous for those women who switch from other OCs or who start an OC for the first time, since these patients are known to often experience frequent intermenstrual bleeding, which may lead to discontinuation of (extended) oral contraceptive use.

The following example shall explain the invention in further detail without limiting the invention to this special example:

### EXAMPLE 1

The following clinical protocol can be performed to examine the bleeding profile obtained for the method of the present invention. This protocol is proposed to demonstrate the superior bleeding properties of the EE/DNG extended cycle oral contraceptive compared to the corresponding standard cycle OC (21+7 days).

A one-year, multi-center, open, randomized, parallel-group comparative phase III study in 1,100 healthy young fertile women (400 evaluable cases per group) of 18-40 years is performed with an OC according to the method of the invention. The OC contains 2 mg of dienogest (DNG) and 30 μg ethinyl estradiol (EE) (CELIMONA®/VALETTE®).

In the present protocol 4 extended cycles of 84 days followed by a 7-day pill-free break (no intake) are compared with 13 conventional 21-day cycles followed by a 7-day pill-free break each. For regulatory reasons all women on the extended cycle are required to adhere to the 84 day schedule in order to obtain sufficient safety exposure/data for the maximum intended duration of use.

Standard inclusion and exclusion criteria for oral contraceptive studies are used.

The primary study parameter is the number of days with intracyclic bleeding during one year.

Additionally bleeding pattern and cycle control parameters are evaluated via a daily paper diary. The minimum goal is to demonstrate a significant reduction in the total number of bleeding days during one year (i.e. 364 days) for the extended cycle (84+7 days) compared to the standard cycle (21+7 days) use.

The number of unintended pregnancies is assessed (Pearl Index, Life table analysis). Additionally standard safety parameters for OCs are studied.

While the invention has been illustrated and described as embodied in a method of female hormonal contraception using dienogest in fixed extended cycle hormonal contraceptives and a pharmaceutical package with instructions for performing the method, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A method for female hormonal contraception, said method comprising continuous daily administration of daily dosage units of a monophasic preparation to a woman over a time period of n×21 days, wherein n=2, 3, 4 or 5, wherein said time period is followed by a seven-day pill-break in which said monophasic preparation is not administered for seven consecutive days immediately following said time period; and
wherein each of said daily dosage units of said monophasic preparation contains from 0.5 to 3.0 mg of dienogest and from 10 to 30 μ of ethinyl estradiol or a synthetic or natural estrogen at a daily dosage equivalent in estrogenic activity to 10 to 30 μg of said ethinyl estradiol.

2. The method according to claim 1, wherein said seven-day pill-break is taken after 42, 63 or 84 days of said continuous daily administration of said daily dosage units of said monophasic preparation.

3. The method according to claim 1, wherein said time period is 84 days or said n is 4.

4. The method according to claim 1, wherein each of said daily dosage units of said monophasic preparation contains from 1.0 to 2.5 mg of said dienogest.

5. The method according to claim 1, wherein each of said daily dosage units of said monophasic preparation contains 2.0 mg of said dienogest.

6. The method according to claim 1, wherein each of said daily dosage units of said monophasic preparation contains from 20 to 30 μg of said ethinyl estradiol or said synthetic or natural estrogen at a daily dosage equivalent in said estrogenic activity to 20 to 30 μg of said ethinyl estradiol.

7. The method according to claim 1, wherein each of said daily dosage units of said monophasic preparation contains 30 μg of said ethinyl estradiol or said synthetic or natural estrogen at a daily dosage equivalent in said estrogenic activity to 30 μg of said ethinyl estradiol.

8. The method according to claim 1, wherein said continuous daily administration to said woman is oral administration and said daily dosage units are oral dosage units.

9. A pharmaceutical package comprising
a number of individual daily dosage units of a monophasic preparation, each of said daily dosage units containing from 0.5 to 3.0 mg of dienogest and from 10 to 30 μ of ethinyl estradiol or a synthetic or natural estrogen at a daily dosage equivalent in estrogenic activity to 10 to 30 μ of said ethinyl estradiol, said number being selected for performing the method for female hormonal contraception according to claim 1 ,and
patient instructions for use of said monophasic preparation according to the method for female hormonal contraception according to said claim 1.

10. A method of fixed extended cycle hormonal contraception and simultaneous reduction of acne produced as a side effect during the hormonal contraception, said method comprising continuous daily administration of daily dosage units of a monophasic preparation to a woman over a time period of n×21 days, wherein n 2, 3, 4 or 5, wherein said time period is followed by a seven-day pill-break in which said monophasic preparation is not administered for seven consecutive days immediately following said time period; and
wherein each of said daily dosage units of said monophasic preparation contains dienogest and ethinyl estradiol.

11. The method according to claim 10, wherein each of said daily dosage units contains from 0.5 to 3.0 mg of said dienogest and from 10 to 30 μ of said ethinyl estradiol or said synthetic or natural estrogen at a daily dosage equivalent in said estrogenic activity to 10 to 30 μg of said ethinyl estradiol.

12. The method according to claim 10, wherein said seven-day pill-break is taken after 42, 63 or 84 days of said continuous daily administration of said daily dosage units of said monophasic preparation.

13. The method according to claim 10, wherein each of said daily dosage units of said monophasic preparation contains from 1.0 to 2.5 mg of said dienogest and from 20 to 30 μ of said ethinyl estradiol or said synthetic or natural estrogen at a daily dosage equivalent in said estrogenic activity to 10 to 30 μg of said ethinyl estradiol.

14. A method of fixed extended cycle hormonal contraception and simultaneous treatment of dysmenorrhea and/or endometriosis side effects of the hormonal contraception, said method comprising continuous daily administration of daily dosage units of a monophasic preparation to a woman over a time period of n×21 days, wherein n=2, 3, 4 or 5, wherein said time period is followed by a seven-day pill-break in which said monophasic preparation is not administered for seven consecutive days immediately following said time period; and
wherein each of said daily dosage units of said monophasic preparation contains dienogest and ethinyl estradiol.

15. The method as defined in claim 14, wherein each of said daily dosage units contains from 0.5 to 3.0 mg of said dienogest and from 10 to 30 μg of said ethinyl estradiol or a synthetic or natural estrogen at a daily dosage equivalent in estrogenic activity to 10 to 30 μ of said ethinyl estradiol.

16. The method according to claim 14, wherein said seven-day pill-break is taken after 42, 63 or 84 days of said continuous daily administration of said daily dosage units of said monophasic preparation.

17. The method according to claim 14, wherein each of said daily dosage units of said monophasic preparation contains from 1.0 to 2.5 mg of said dienogest and from 20 to 30 μg of said ethinyl estradiol or a synthetic or natural estrogen at a daily dosage equivalent in estrogenic activity to 10 to 30 μg of said ethinyl estradiol.

18. A method of treating acne, said method comprising continuous daily administration of daily dosage units of a monophasic preparation to a woman over a time period of n×21 days, wherein n=2, 3, 4 or 5, wherein said time period is followed by a seven-day pill-break in which said monophasic preparation is not administered for seven consecutive days immediately following said time period; and
wherein each of said daily dosage units of said monophasic preparation contains from 0.5 to 3.0 mg of dienogest and from 10 to 30 μg of ethinyl estradiol or a synthetic or natural estrogen at a daily dosage equivalent in estrogenic activity to 10 to 30 μ of said ethinyl estradiol.

19. A method of treating dysmenorrhea, said method comprising continuous daily administration of daily dosage units of a monophasic preparation to a woman over a time period of n×21 days, wherein n=2, 3, 4 or 5, wherein said time period is followed by a seven-day pill-break in which said monophasic preparation is not administered for seven consecutive days immediately following said time period; and
wherein each of said daily dosage units of said monophasic preparation contains from 0.5 to 3.0 mg of dienogest and from 10 to 30 μ of ethinyl estradiol or a synthetic or natural estrogen at a daily dosage equivalent in estrogenic activity to 10 to 30 μg of said ethinyl estradiol.

20. A method of treating endometriosis, said method comprising continuous daily administration of daily dosage units of a monophasic preparation to a woman over a time period of n×21 days, wherein n=2, 3, 4 or 5, wherein said time period is followed by a seven-day pill-break in which said monophasic preparation is not administered for seven consecutive days immediately following said time period; and
wherein each of said daily dosage units of said monophasic preparation contains from 0.5 to 3.0 mg of dienogest and from 10 to 30 μg of ethinyl estradiol or a synthetic or natural estrogen at a daily dosage equivalent in estrogenic activity to 10 to 30 μg of said ethinyl estradiol.
